(19) United States
(12) Patent Application Publication
(10) Pub. No.: US 2021/0349305 A1
(43) Pub. Date: Nov. 11, 2021

(54) OPTICAL TRANSDUCER FOR ENDOSCOPE, IMAGE PICKUP APPARATUS FOR ENDOSCOPE, AND ENDOSCOPE

(71) Applicant: OLYMPUS CORPORATION, Tokyo (JP)

(72) Inventors: Takahide MIYAWAKI, Kamiina-gun (JP); Hiroyuki MOTOHARA, Tokyo (JP); Yohei SAKAI, Ina-shi (JP)

(73) Assignee: OLYMPUS CORPORATION, Tokyo (JP)

(21) Appl. No.: 17/380,674

(22) Filed: Jul. 20, 2021

Related U.S. Application Data
(63) Continuation of application No. PCT/JP2019/009172, filed on Mar. 7, 2019.

(51) Int. Cl. G02B 23/26; H04N 5/225

(52) U.S. Cl. CPC G02B 23/26; A61B 1/00096; H04N 2005/2255; H04N 5/2253

(57) ABSTRACT

An image pickup apparatus for an endoscope includes: a light emitting element configured to emit an optical signal; an optical fiber configured to transmit the optical signal; metal cables configured to transmit an electric signal; a ferrule including an insertion hole into which the optical fiber is inserted; and a molded interconnect device to which core wires of the metal cables and the light emitting element are electrically connected, and which is integrally molded, the molded interconnect device including a recess in which the light emitting element and the ferrule are accommodated.

### CROSS REFERENCE TO RELATED APPLICATION

This application is a continuation application of PCT/JP2019/009172 filed on Mar. 7, 2019, the entire contents of which are incorporated herein by this reference.

### BACKGROUND OF THE INVENTION

### 1. Field of the Invention

The present invention relates to an optical transducer for an endoscope, the optical transducer including a light emitting element, a ferrule, an optical fiber, and a molded interconnect device. The present invention also relates to an image pickup apparatus for an endoscope, the image pickup apparatus including an optical transducer for an endoscope, the optical transducer including a light emitting element, a ferrule, an optical fiber, and a molded interconnect device. The present invention further relates to an endoscope which includes an image pickup apparatus for an endoscope, the image pickup apparatus including an optical transducer for an endoscope, the optical transducer including a light emitting element, a ferrule, an optical fiber, and a molded interconnect device.

### 2. Description of the Related Art

An endoscope includes an image pickup member which includes an image pickup device at a distal end portion of an elongated insertion portion. In recent years, the use of an image pickup device having a large number of pixels in an endoscope has been considered. In an endoscope which uses an image pickup device having a large number of pixels, an amount of signals transmitted to a signal processing device from the image pickup device increases. Therefore, it is preferable to adopt optical signal transmission, where optical signals are transmitted via optical fibers, in place of electric signal transmission, where eclectic signals are transmitted via metal wires. For the optical signal transmission, an E/O optical transducer (electric-to-optical transducer) and an O/E optical transducer (optical-to-electric transducer) are used. The E/O optical transducer converts an electric signal to an optical signal. The O/E optical transducer converts an optical signal to an electric signal.

Japanese Patent Application Laid-Open Publication No. 2013-025092 discloses an optical transducer which includes a light emitting element and a ferrule, the light emitting element generating optical signals, the ferrule having an insertion hole into which an optical fiber, which transmits the optical signals, is inserted.

To achieve a minimally-invasive endoscope, it is important to reduce the size of an optical transducer, that is, to reduce a diameter and a length of the optical transducer. Further, not only an optical fiber but also a metal cable which transmits electric signals are connected to the optical transducer.

### SUMMARY OF THE INVENTION

An aspect is directed to an optical transducer for an endoscope, the optical transducer including: at least one light emitting element configured to emit an optical signal; at least one optical fiber configured to transmit the optical signal; a plurality of metal cables configured to transmit an electric signal; a ferrule including at least one insertion hole into which the at least one optical fiber is inserted; and a molded interconnect device to which core wires of the plurality of metal cables and the at least one light emitting element are electrically connected, and which is integrally molded, the molded interconnect device including a recess in which the at least one light emitting element and the ferrule are accommodated.

Another aspect is directed to an optical transducer for an endoscope, the optical transducer including: at least one light emitting element configured to emit an optical signal; at least one optical fiber configured to transmit the optical signal; a plurality of metal cables configured to transmit an electric signal; a ferrule including at least one insertion hole into which the at least one optical fiber is inserted; and a molded interconnect device to which core wires of the plurality of metal cables and the at least one light emitting element are electrically connected, and which has a recess in which the at least one light emitting element and the ferrule are accommodated, wherein the molded interconnect device includes a first molded interconnect device and a second molded interconnect device, the first molded interconnect device including a first main surface and a second main surface on an opposite side to the first main surface, the first main surface including the recess, the second molded interconnect device including a third main surface and a fourth main surface on an opposite side to the third main surface, the third main surface facing the second main surface, the first molded interconnect device includes a plurality of first connection portions molded on the second main surface by insertion molding, the second molded interconnect device includes a plurality of second connection portions molded on the third main surface by insertion molding, and each of the plurality of first connection portions and each of the plurality of second connection portions are fitted to each other, thus causing the first molded interconnect device and the second molded interconnect device to be electrically connected with each other.

Still another aspect is directed to an image pickup apparatus for an endoscope, the image pickup apparatus including an optical transducer for an endoscope, an image pickup device, a plurality of electronic components, and a wiring board on which the image pickup device, the plurality of electronic components, and the molded interconnect device are mounted, the optical transducer for an endoscope including at least one light emitting element configured to emit an optical signal, at least one optical fiber configured to transmit the optical signal, a plurality of metal cables configured to transmit an electric signal, a ferrule including at least one insertion hole into which the at least one optical fiber is inserted, and a molded interconnect device to which core wires of the plurality of metal cables and the at least one light emitting element are electrically connected, and which is integrally molded, the molded interconnect device including a recess in which the at least one light emitting element and the ferrule are accommodated.

Still another aspect is directed to an endoscope including an image pickup apparatus for an endoscope, wherein the image pickup apparatus for an endoscope includes an optical transducer for an endoscope, an image pickup device, a plurality of electronic components, and a wiring board on which the image pickup device, the plurality of electronic components, and the molded interconnect device are mounted, the optical transducer for an endoscope including at least one light emitting element configured to emit an optical signal, at least one optical fiber configured to transmit the optical signal, a plurality of metal cables configured to transmit an electric signal, a ferrule including at least one insertion hole into which the at least one optical fiber is inserted, and a molded interconnect device to which core wires of the plurality of metal cables and the at least one light emitting element are electrically connected, and which is integrally molded, the molded interconnect device including a recess in which the at least one light emitting element and the ferrule are accommodated.

### BRIEF DESCRIPTION OF THE DRAWINGS

FIG. 1 is a configuration diagram of an endoscope system including an endoscope of an embodiment;

FIG. 2 is a perspective view of an image pickup apparatus for an endoscope of a first embodiment;

FIG. 3 is a perspective exploded view of the image pickup apparatus for an endoscope of the first embodiment:

FIG. 4A is a perspective view of another molded interconnect device of an optical transducer of the first embodiment:

FIG. 4B is a perspective view of another molded interconnect device of the optical transducer of the first embodiment;

FIG. 4C is a perspective view of another molded interconnect device of the optical transducer of the first embodiment;

FIG. 5 is a perspective view of an image pickup apparatus for an endoscope of a second embodiment;

FIG. 6 is a perspective exploded view of an optical transducer of the second embodiment;

FIG. 7 is a cross-sectional exploded view of the optical transducer of the second embodiment;

FIG. 8 is a back view of the optical transducer of the second embodiment; and

FIG. 9 is a back view of another optical transducer of the second embodiment.

### DETAILED DESCRIPTION OF THE PREFERRED EMBODIMENTS

### <Endoscope System>

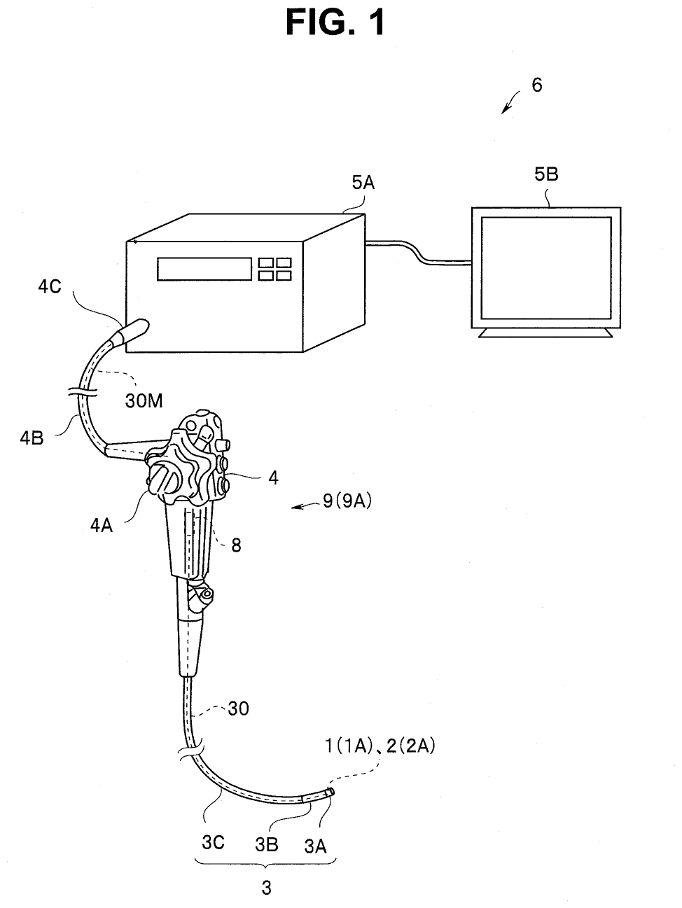

An endoscope 9, 9A of an embodiment shown in FIG. 1 forms an endoscope system 6 in conjunction with a processor 5A and a monitor 5B.

The endoscope 9 includes an insertion portion 3, a grasping portion 4, a universal cord 4B, and a connector 4C. The grasping portion 4 is provided at a proximal end portion of the insertion portion 3. The universal cord 4B extends from the grasping portion 4. The connector 4C is provided at a proximal end portion of the universal cord 4B. The insertion portion 3 includes a distal end portion 3A, a bending portion 3B, and a flexible portion 3C. The bending portion 3B extends from the distal end portion 3A and is bendable to change a direction of the distal end portion 3A. The flexible portion 3C extends from the bending portion 3B. The grasping portion 4 is provided with a rotatable angle knob, 4A which is an operation portion provided to allow an operator to operate the bending portion 3B.

The universal cord 4B is connected to the processor 5A by the connector 4C. The processor 5A controls the entire endoscope system 6, and performs signal processing on an image pickup signal, and outputs the image pickup signal as an image signal. The monitor 5B displays the image signal, which is outputted by the processor 5A, as an endoscope image. The endoscope 9 is a flexible endoscope. However, the endoscope 9 may be a rigid endoscope. The endoscope 9 may be used as a medical endoscope or an industrial endoscope.

An image pickup apparatus for endoscope 1, 1A (hereinafter referred to as “image pickup apparatus”) of the embodiment includes an optical transducer for endoscope 2, 2A (hereinafter referred to as “optical transducer”), and is disposed at the distal end portion 3A of the endoscope 9, 9A.

The optical transducer 2 converts an image pickup signal to an optical signal. An optical signal passes through an optical fiber 30, which is inserted through the insertion portion 3, and is then converted to an electric signal again by an O/E optical transducer 8, which is disposed at the grasping portion 4. The electric signal is transmitted via an electric cable 30M which is inserted through the universal cord 4B. That is to say, in the small-diameter insertion portion 3, an image pickup signal is transmitted via the optical fiber 30. In the universal cord 4B, which is not inserted into the body, and has little restriction on an outer diameter, an image pickup signal is transmitted via the electric cable 30M having a larger diameter than the optical fiber 30.

In the case where the optical transducer 8 is disposed in the connector 4C, the optical fiber 30 is inserted through the universal cord 4B.

The optical transducer 8 is disposed in the grasping portion 4 having a relatively large arrangement space. However, the optical transducer 8 may have the same configuration as the optical transducer of the image pickup apparatus 1.

As will be described later, the image pickup apparatus 1, 1A has a small size and can be easily manufactured. Therefore, the endoscope 9, 9A is minimally invasive and can also be easily manufactured.

### First Embodiment

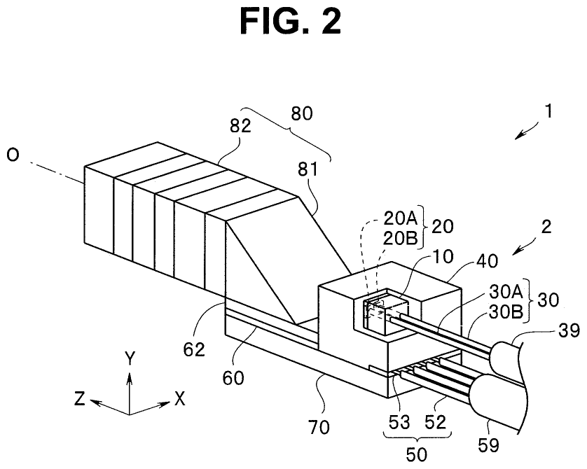

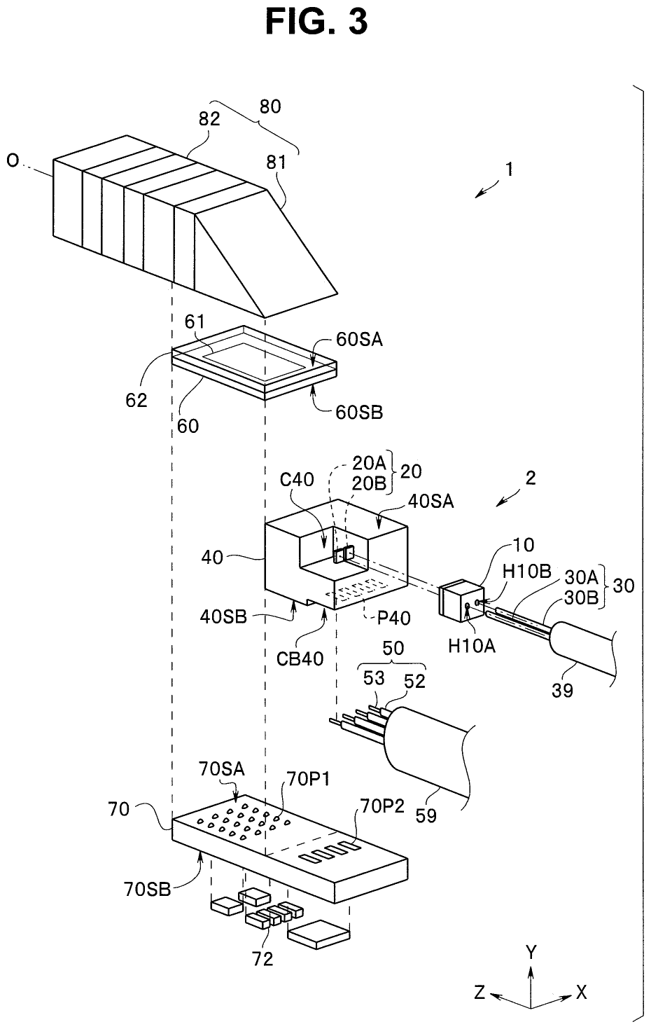

The image pickup apparatus 1 and the optical transducer 2 of a first embodiment will be described with reference to FIG. 2 and FIG. 3.

In the description made hereinafter, drawings based on respective embodiments are schematic views. A relationship between thicknesses and widths of the respective portions, or a ratio between thicknesses of the respective portions, for example, may differ from actual ones. The dimensional relations and the ratio may be partially different between drawings. The illustration of and use of reference symbols for some constitutional elements may be omitted.

The image pickup apparatus 1 includes the optical transducer 2, an image pickup device 60, a wiring board 70, and an optical system 80. The optical transducer 2 includes a ferrule 10, light emitting elements 20A, 20B, optical fibers 30A, 30B, a molded interconnect device 40, and a plurality of metal cables 50.

Hereinafter, when each of a plurality of constitutional elements is referred to, one letter at the end of reference symbol may be omitted. For example, each of the light emitting elements 20A, 20B is referred to as “light emitting element 20”. Further, a direction in which the molded interconnect device 40 is disposed on the wiring board 70 (a direction of increasing values of a Y axis in the drawing) is referred to as “upward”, and a direction opposite to “upward” (a direction of decreasing values of the Y axis in the drawing) is referred to as “downward”.

The optical system 80 includes a right-angle prism 81 and a plurality of optical elements 82. A subject image which is focused by the plurality of optical elements 82 passes through the right-angle prism 81 and a cover glass 62, and is incident on a light receiving unit 61 on a light receiving surface 60SA of the image pickup device 60 which is orthogonal to an optical axis O. The optical system 80 may be separated from the image pickup apparatus 1, or may be combined with an optical system which is a separate member.

The image pickup device 60 is a CCD chip or a COMS image pickup chip which includes the light receiving unit 61. The cover glass 62, which protects the light receiving unit 61, is caused to adhere to the light receiving surface 60SA of the image pickup device 60. A plurality of external electrodes (not shown in the drawing) connected to the light receiving unit 61 are provided to a rear surface 60SB on the opposite side to the light receiving surface 60SA.

The wiring board 70 uses FPC, ceramic, glass epoxy, glass, silicon, or the like as a base body. A plurality of first electrodes 70P1 on a front surface 70SA are bonded to the external electrodes of the image pickup device 60. A plurality of second electrodes 70P2 on the front surface 70SA are bonded to lower surface electrodes (not shown in the drawing) of the molded interconnect device 40. Each of a plurality of third electrodes (not shown in the drawing) on a rear surface 70SB on the opposite side to the front surface 70SA, is bonded to each of a plurality of electronic components 72, such as a chip capacitor or a drive IC.

That is to say, the image pickup device 60, the molded interconnect device 40, and the plurality of electronic components 72 are mounted on the wiring board 70.

An image pickup signal outputted by the image pickup device 60 is, by the electronic components 72, converted to a drive signal which drives the light emitting element 20.

The ferrule 10 has insertion holes H10A, H10B into which the optical fibers 30A, 30B are respectively inserted. For example, the ferrule 10 is formed of a silicon substrate having the insertion holes H10, and a glass substrate which is bonded to the silicon substrate. Each light emitting element 20 is mounted on the glass substrate of the ferrule 10.

The light emitting element 20 may be a surface emitting laser or the like which outputs light for an optical signal. For example, the extremely-small light emitting element 20, with dimensions of 250 μm×250 μm as viewed in a plan view, includes a light emitting unit and an external electrode on a light emitting surface which are not shown in the drawing. The light emitting unit has a diameter of 10 μm. The external electrode supplies a drive signal to the light emitting unit.

The light emitting elements 20A, 20B are disposed in a state where the light emitting units of the light emitting elements 20A, 20B respectively face the insertion holes H10A, H10B of the ferrule 10.

For example, each of the plurality of optical fibers 30 is formed of a core and a clad, the core having a diameter of 50 μm and transmitting light, the clad having a diameter of 125 μm and covering an outer periphery of the core. Fiber rear portions of the plurality of optical fibers 30 are accommodated in one protective tube 39.

The molded interconnect device (MID) 40 is an electrical component where electrical elements, such as wiring and connectors, are formed into an integral body with a structural member, which forms a housing or the like, by using techniques such as injection molding and metal plating on plastic. The molded interconnect device (MID) 40 has both a mechanical function and an electrical function.

The molded interconnect device 40 having a substantially rectangular parallelepiped shape is integrally molded, and includes an upper surface 40SA and a lower surface 40SB on the opposite side to the upper surface 40SA. The molded interconnect device 40 has a recess C40 which has an opening on the upper surface 40SA and two side surfaces orthogonal to the upper surface 40SA.

The light emitting elements 20 and the ferrule 10 are completely accommodated in the recess C40.

The light emitting elements 20 and the ferrule 10 are completely accommodated in the recess C40 of the molded interconnect device 40 having a wiring function and hence, the optical transducer 2 has small outer dimensions in a direction of the optical axis and a direction orthogonal to the optical axis.

Each metal cable 50 includes a core wire 53 and an insulation layer 52, the core wire 53 being made of a conductor, the insulation layer 52 covering the core wire 53. Cable rear portions of the plurality of metal cables 50 are accommodated in one cover tube 59.

The protective tube 39 and the cover tube 59 are disposed adjacent and parallel to each other.

As shown in FIG. 7, a length L30 of fiber front portions of the plurality of optical fibers 30 protruding from the protective tube 39 is larger than a length L50 of cable front portions of the plurality of metal cables 50 protruding from the cover tube 59.

If the optical fiber 30 is significantly deformed, the optical fiber 30 may break. In contrast, even if the metal cable 50 is significantly deformed, there is a low chance of breakage compared with the optical fiber 30. When the length L30 of the fiber front portion is set to be larger than the length L50 of the cable front portion, an amount of deformation of the optical fiber 30 is smaller than an amount of deformation of the metal cable 50, thus lowering the chance of breaking the optical fiber 30.

A cutout CB40 is formed on the lower surface 40SB of the molded interconnect device 40. Electrodes P40 are disposed on the cutout CB40. The core wires 53 of the metal cables 50 are respectively bonded to the electrodes P40. The core wires 53 of the metal cables 50 are sandwiched between the cutout CB40 on the lower surface 40SB and the front surface 70SA of the wiring board 70. Therefore, the core wires 53 of the optical transducer 2 have a high reliability of bonding. A gap formed between the cutout CB40 and the front surface 70SA of the wiring board 70 may be filled with a resin.

The ferrule 10, on which the light emitting elements 20 are mounted, is mounted on the molded interconnect device 40. The light emitting elements 20 may be mounted on an inner surface of the recess C40.

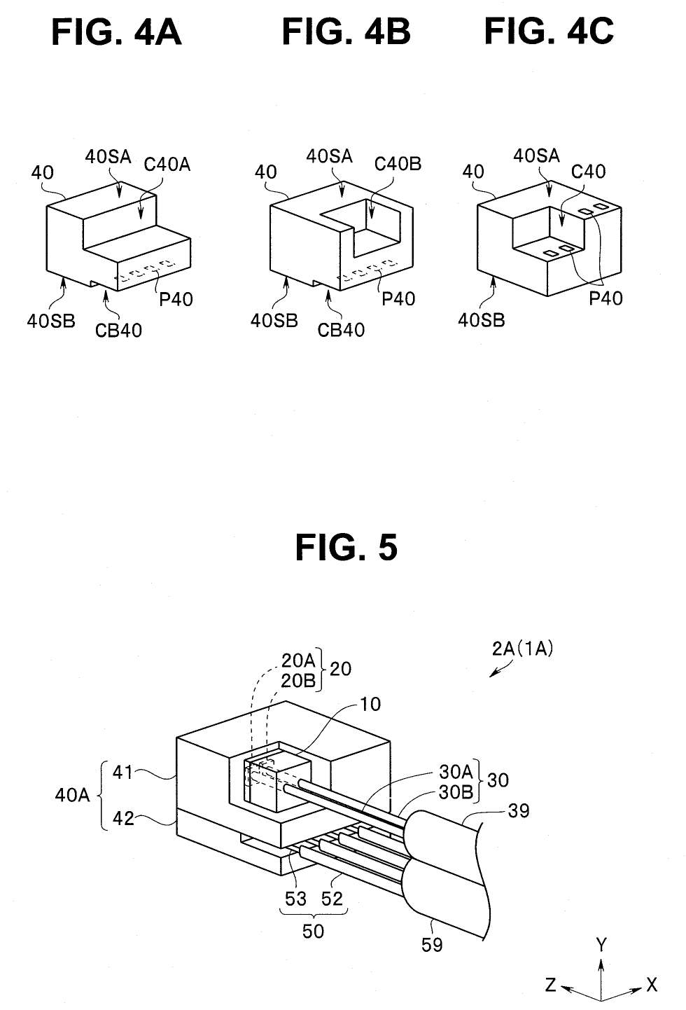

The molded interconnect device 40 may have a configuration shown in FIG. 4A where the molded interconnect device 40 has a recess C40A which completely accommodates the light emitting elements 20 and the ferrule 10. Alternatively, the molded interconnect device 40 may have a configuration shown in FIG. 4B where the molded interconnect device 40 has a recess C40B which completely accommodates the light emitting elements 20 and the ferrule 10. Further, as shown in FIG. 4C, the electrodes P40 to which the core wires 53 of the metal cables 50 are respectively bonded may be provided to at least either one of the recess C40 or the upper surface 40SA.

### Second Embodiment

An image pickup apparatus 1A and an optical transducer 2A of a second embodiment are similar to and have substantially the same advantageous effects as the image pickup apparatus 1 and the optical transducer 2 and hence, constitutional elements having the same function are given the same reference symbols, and the repeated description will be omitted.

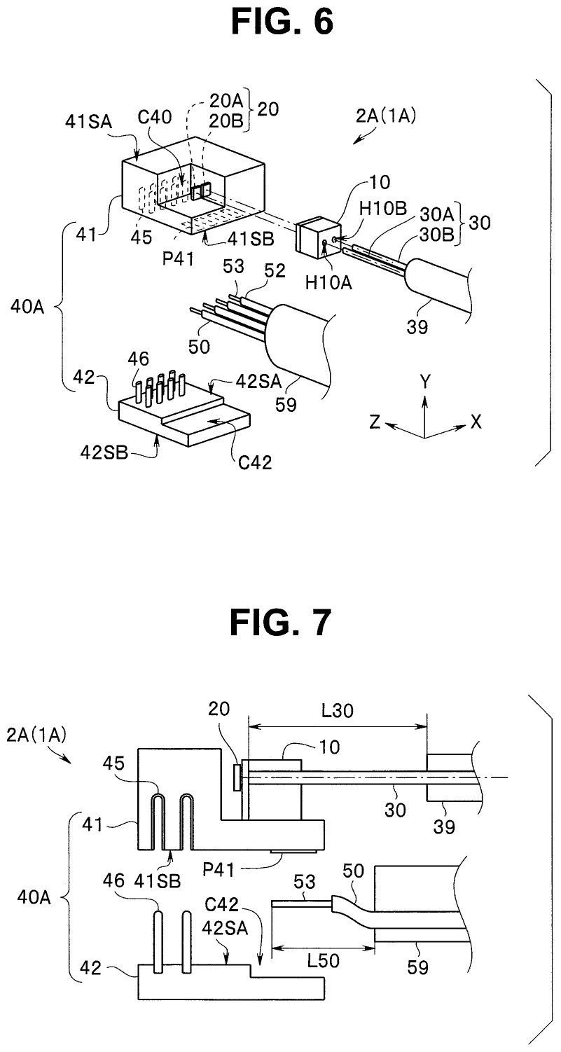

In the optical transducer 2A of the image pickup apparatus 1A shown in FIG. 5 to FIG. 7, a molded interconnect device 40A includes a first molded interconnect device 41 and a second molded interconnect device 42.

The first molded interconnect device 41 includes a first main surface 41SA and a second main surface 41SB on the opposite side to the first main surface 41SA. The second molded interconnect device 42 includes a third main surface 42SA and a fourth main surface 42SB on the opposite side to the third main surface 42SA.

The first molded interconnect device 41 includes a plurality of first connection portions 45 formed on the second main surface 41SB by insertion molding. Electrodes P41 are provided to the second main surface 41SB. The second molded interconnect device 42 includes a plurality of second connection portions 46 formed on the third main surface 42SA by insertion molding.

In insertion molding, for example, the plurality of first connection portions 45 are disposed in a cavity of a molding die. In this state, a molten resin is injected and filled into the cavity to integrally mold the resin and the plurality of first connection portions 45.

The second main surface 41SB and the third main surface 42SA are disposed to face each other, and the first connection portions 45 and the second connection portions 46 are fitted with each other, the first connection portions 45 being recessed electrodes, the second connection portions 46 being protruding electrodes. With such a configuration, the first molded interconnect device 41 and the second molded interconnect device 42 are formed into an integral body, and are electrically connected with each other.

An upper surface of the molded interconnect device 40A is formed of the first main surface 41SA of the first molded interconnect device 41, and a lower surface of the molded interconnect device 40A is formed of the fourth main surface 42SB of the second molded interconnect device 42.

In a method for manufacturing the image pickup apparatus 1A, the light emitting elements 20 are mounted on the ferrule 10 and, thereafter, the ferrule 10 is mounted on the recess C40 of the first molded interconnect device 41. The core wires 53 of the cables 50 are bonded to the electrodes P41 on the second main surface 41SB of the first molded interconnect device 41. The optical fibers 30 are inserted into and fixed to the insertion holes H10 of the ferrule 10.

In contrast, the image pickup device 60, the second molded interconnect device 42 and the like are mounted on the wiring board 70.

The image pickup apparatus 1A is prepared by causing the first connection portions 45 and the second connection portions 46 to be fitted with each other. Therefore, the image pickup apparatus 1A can be easily manufactured.

The first connection portions 45 may be protruding electrodes. The second connection portions 46 may be recessed electrodes. For example, the plurality of first connection portions 45 may include protruding electrodes and recessed electrodes.

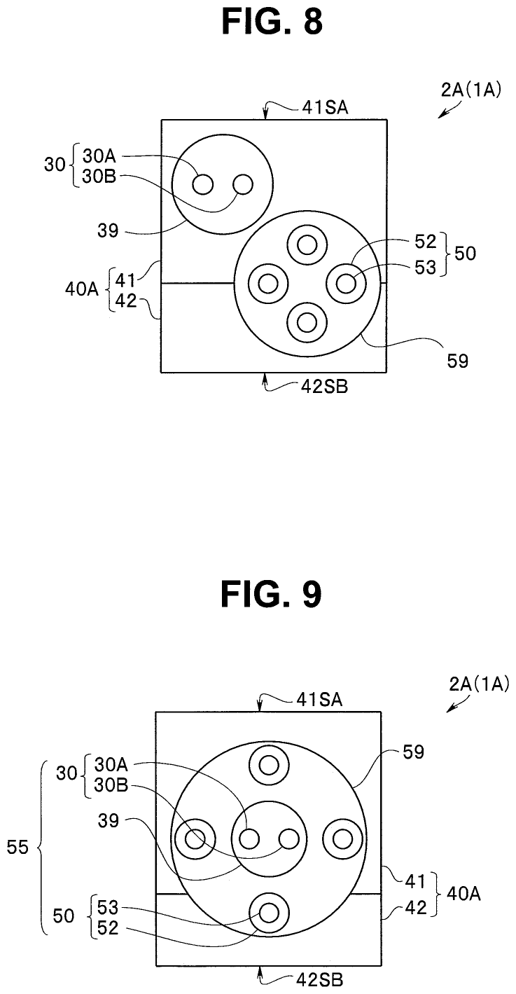

As shown in FIG. 8, a distal end portion of the protective tube 39 and a distal end portion of the cover tube 59 are accommodated in spaces, the spaces extending from the molded interconnect device 40A in a longitudinal direction of the distal end portions. Therefore, an outer dimension of the optical transducer 2A can be reduced.

As shown in FIG. 9, the optical transducer 2A may include a photoelectric composite cable 55 which includes the optical fiber 30 and the metal cable 50.

That is to say, the present invention is not limited to the above-mentioned embodiments or modifications, and various changes, combinations, and applications are conceivable without departing from the gist of the present invention.

What is claimed is:

1. An optical transducer for an endoscope, the optical transducer comprising:
at least one light emitting element configured to emit an optical signal:
at least one optical fiber configured to transmit the optical signal;
a plurality of metal cables configured to transmit an electric signal;
a ferrule including at least one insertion hole into which the at least one optical fiber is inserted; and
a molded interconnect device to which core wires of the plurality of metal cables and the at least one light emitting element are electrically connected, and which is integrally molded, the molded interconnect device including a recess in which the at least one light emitting element and the ferrule are accommodated.

2. An optical transducer for an endoscope, the optical transducer comprising:
at least one light emitting element configured to emit an optical signal;
at least one optical fiber configured to transmit the optical signal;
a plurality of metal cables configured to transmit an electric signal;
a ferrule including at least one insertion hole into which the at least one optical fiber is inserted; and
a molded interconnect device to which core wires of the plurality of metal cables and the at least one light emitting element are electrically connected, and which has a recess in which the at least one light emitting element and the ferrule are accommodated, wherein
the molded interconnect device includes a first molded interconnect device and a second molded interconnect device, the first molded interconnect device including a first main surface and a second main surface on an opposite side to the first main surface, the first main surface including the recess, the second molded interconnect device including a third main surface and a fourth main surface on an opposite side to the third main surface, the third main surface facing the second main surface,
the first molded interconnect device includes a plurality of first connection portions formed on the second main surface by insertion molding,
the second molded interconnect device includes a plurality of second connection portions formed on the third main surface by insertion molding, and
each of the plurality of first connection portions and each of the plurality of second connection portions are fitted to each other, thus causing the first molded interconnect device and the second molded interconnect device to be electrically connected with each other.

3. The optical transducer for an endoscope according to claim 2, wherein
each of the core wires of the plurality of metal cables is sandwiched between the second main surface and the third main surface.

4. The optical transducer for an endoscope according to claim 1, the optical transducer comprising
a plurality of light emitting elements and a plurality of optical fibers, wherein
fiber rear portions of the plurality of optical fibers are accommodated in one protective tube,
cable rear portions of the plurality of metal cables are accommodated in one cover tube, and
the protective tube and the cover tube are disposed adjacent and parallel to each other.

5. The optical transducer for an endoscope according to claim 4, wherein
a length of fiber front portions of the plurality of optical fibers protruding from the protective tube is larger than a length of cable front portions of the plurality of metal cables protruding from the cover tube.

6. The optical transducer for an endoscope according to claim 4 comprising a photoelectric composite cable including the protective tube and the cover tube.

7. The optical transducer for an endoscope according to claim 6, wherein
each of a distal end portion of the protective tube and a distal end portion of the cover tube is accommodated in a space, the space extending from the molded interconnect device in a longitudinal direction of each distal end portion.

8. An image pickup apparatus for an endoscope, the image pickup apparatus comprising:
an optical transducer for an endoscope, the optical transducer including at least one light emitting element configured to emit an optical signal, at least one optical fiber configured to transmit the optical signal, a plurality of metal cables configured to transmit an electric signal, a ferrule including at least one insertion hole into which the at least one optical fiber is inserted, and a molded interconnect device to which core wires of the plurality of metal cables and the at least one light emitting element are electrically connected, and which is integrally molded, the molded interconnect device including a recess in which the at least one light emitting element and the ferrule are accommodated;
an image pickup device;
a plurality of electronic components; and
a wiring board on which the image pickup device, the plurality of electronic components, and the molded interconnect device are mounted.

9. An endoscope comprising an image pickup apparatus for an endoscope, wherein
the image pickup apparatus for an endoscope includes:
an optical transducer for an endoscope, the optical transducer including at least one light emitting element configured to emit an optical signal, at least one optical fiber configured to transmit the optical signal, a plurality of metal cables configured to transmit an electric signal, a ferrule including at least one insertion hole into which the at least one optical fiber is inserted, and a molded interconnect device to which core wires of the plurality of metal cables and the at least one light emitting element are electrically connected, and which is integrally molded, the molded interconnect device including a recess in which the at least one light emitting element and the ferrule are accommodated;
an image pickup device;
a plurality of electronic components; and
a wiring board on which the image pickup device, the plurality of electronic components, and the molded interconnect device are mounted.
